(19) United States
(12) Patent Application Publication
(10) Pub. No.: US 2004/0053315 A1
(43) Pub. Date: Mar. 18, 2004

(54) Methods and systems for monitoring molecular interactions

(75) Inventors: Michael Spaid, Mountain View, CA (US); Jeffrey A. Wolk, Half Moon Bay, CA (US)

Correspondence Address: CALIPER TECHNOLOGIES CORP, 605 FAIRCHILD DRIVE, MOUNTAIN VIEW, CA, 94043, US

(73) Assignee: Caliper Technologies Corp., Mountain View, CA

(21) Appl. No.: 10/634,742

(22) Filed: Aug. 4, 2003

Related U.S. Application Data
(60) Provisional application No. 60/402,508, filed on Aug. 12, 2002.

(51) Int. Cl. C12Q 1/68; G01N 33/543

(52) U.S. Cl. 435/6; 436/518

(57) ABSTRACT

The present invention relates to novel methods and systems for determining the interaction of molecules using the phenomena of Taylor-Aris dispersion present in fluid flow in conduits. The method involves relating a change in dispersion of molecules to their level of interaction. The present invention also relates to an assay method using Taylor-Aris dispersion in a microfluidic system in order to examine molecular interactions in a variety of chemical and biochemical systems.

### CROSS-REFERENCE TO RELATED APPLICATIONS

This application claims the benefit of U.S. Provisional Patent Application No. 60/402,508, filed Aug. 12, 2002, which is incorporated herein by reference in its entirety for all purposes.

### STATEMENT REGARDING FEDERALLY SPONSORED RESEARCH AND DEVELOPMENT

Not applicable.

### TECHNICAL FIELD OF THE INVENTION

This application discloses novel methods and systems for monitoring molecular interactions or associations using changes in physical properties of the molecules in flowing fluidic systems, such as, e.g., rates of Taylor-Aris dispersion. The invention generally relates to methods of observing changes in levels of association between molecules in fluidic conduits, and preferably, microfluidic channel networks.

### BACKGROUND OF THE INVENTION

Recent efforts have been directed to the development of microscale assay methods in which various chemical and biological processes may be examined. Of particular interest are microfluidic chips which use minute quantities of fluids, or other materials, controllably flowed and/or directed, to generate highly reproducible and rapidly changeable microenvironments for control of chemical and biological reaction conditions, enzymatic processes and the like.

Several methods have been developed using microfluidics that are capable of detecting the presence of or interactions between molecules in an analyte solution. The primary method for measuring non-reactive interactions, such as binding, of analytes in solution has been through the use of labels or tags in a heterogenous format. Briefly, a labeled analyte is contacted with a prospective binding partner. The bound label is then separated from any free, e.g., unbound, label in a separation step, such as by chromatography, electrophoresis, or by tethering one or the other component to a solid support followed by a washing step. The disadvantage of these heterogenous formats is that they require additional time and labor-intensive steps.

In some cases, labels are available that produce a signal which becomes modulated when a molecular interaction has occurred. However, measurement of the interaction or reaction processes has been complicated by the fact that many analytes of interest (macromolecules including proteins, polynucleotides, polysaccharide and especially small molecules) either (1) do not have a readily available label that produces a signal only when subjected to the reaction of interest, or (2) labeling of the analyte interferes with the molecular interaction.

Furthermore, for many reactions it is apparent that even when one molecule of an interacting pair is labeled, formation of a complex does not give rise to a detectable difference between the complex and the labeled molecule alone. Therefore, the molecules of many reactions that are of great interest to the biological research field cannot be modified so as to be readily detected by conventional means. In an attempt to solve these problems, researchers have developed several methods which give rise to changes in optical properties upon association of the analytes.

For example, Pirrung et al. (U.S. Pat. No. 5,143,854) describes techniques utilizing the immobilization of one molecule of a binding pair. The labeled molecule is then contacted with the immobilized molecule, and the immobilizing support is washed. The support is then examined for the presence of the labeled molecule, indicating binding of the labeled component to the unlabeled, immobilized component. Vast arrays of different binding member pairs are often prepared in order to enhance the throughput of the assay format.

Alternatively, in the case of nucleic acid hybridization assays, researchers have developed complementary labeling systems that take advantage of the proximity of bound elements to produce fluorescent signals, either in the bound or unbound state. See, e.g., U.S. Pat. Nos. 5,668,648; 5,707,804; 5,728,528; 5,853,992 and 5,869,255 to Mathies et al. for a description of FRET dyes, and Tyagi et al. Nature Biotech. 14:303-8 (1996), and Tyagi et al., Nature Biotech. 16:49-53 (1998) for a description of molecular beacons.

Further, Yamauchi et al. (U.S. Pat. No. 5,723,345) discloses specific binding assay methods by which substances in a liquid sample flow through a channel and interact with a signal substance to generate a signal which is detected by a plurality of detectors.

Maracas, G. N. (U.S. Pat. No. 6,048,691) discloses chip-based molecular detection devices and methods and systems for performing binding assays.

Another homogenous method of detecting binding is through the use of fluorescence polarization. In fluorescence polarization detection, binding of a larger molecule to a small labeled molecule results in a change in the rotational diffusion rate of the labeled species, and thus impedes its ability to emit polarized fluorescence in response to polarized activation energy. See, e.g., U.S. Pat. No. 6,287,774 to Nikiforov.

It is apparent from the forgoing references that most conventional techniques involve the presence of a detection agent or material or the ability of the substrate to bind an agent to produce the detectable signal. The methods may have several drawbacks, including the lack of optical properties of the subject molecules, the potential for interference by the detection agent or label with the binding or molecular association that is the subject of the experiment, and even the lack of suitable labels for reporting a binding event. A common problem with methods of the prior art is that a labeled or substrate-bound molecule may not exhibit the identical binding characteristics that its free counterpart would. By labeling or linking a molecule to a fixed detection substrate, the molecular morphology, binding site availability or accessibility may change, thereby causing inaccurate measurements of its binding characteristics with other molecules.

Accordingly, there is a need for an assay detection method that does not (1) rely on labels that generate a discernible signal upon the occurrence of molecular association or (2) require a separation step following a molecular association to separate free from bound labels.

### SUMMARY OF THE INVENTION

The present invention utilizes the phenomenon of Taylor-Aris dispersion to meet these needs. Although the Taylor-Aris phenomenon has been previously identified (see, e.g., Taylor, Sir Geoffrey, F. R. S., Dispersion of soluble matter in solvent flowing slowly through a tube, Proc. Roy. Soc. (London) 219A:186-203 (1953); Taylor, Sir Geoffrey, F. R. S., Conditions under which dispersion of a solute in a stream of solvent can be used to measure molecular diffusion, Proc. Roy. Soc. (London) A225:473-477 (1954); Aris, R., On the dispersion of a solute in a fluid flowing through a tube, Proc. Roy. Soc. (London) A235:67-77 (1956)), methods and devices involving this process to determine interactions between a plurality of molecules have not been previously described.

In an embodiment, the invention provides a method for determining an interaction between a plurality of molecules. The method comprises flowing a plurality of the molecules in a fluidic conduit, wherein the flow is a pressure-driven flow; measuring the dispersion of at least one of the molecules, wherein the dispersion of the molecules is Taylor-Aris dispersion; and relating the dispersion to the interaction between the plurality of molecules.

In another embodiment, the invention provides a method for determining an interaction between a plurality of molecules. The method comprises introducing a first molecule of a plurality of molecules into a microfluidic conduit; introducing a second molecule of the plurality of molecules into the microfluidic conduit; measuring the dispersion of at least one of the first and second molecules flowing in the microfluidic conduit under pressure-driven flow conditions; and relating the dispersion to the interaction between the plurality of molecules.

In another embodiment, the invention provides a microfluidic system. The system comprises a microfluidic device having a body structure including a first channel and a second channel formed therein, wherein the first and second channels intersect; a fluid sample inlet through which a sample is delivered to the first channel and the second channel; a first fluid reservoir in fluid communication with the first channel, the first channel having an inlet through which a first fluid is delivered from the first reservoir to the first channel; a second fluid reservoir in fluid communication with the second channel, the second channel having an inlet through which a second fluid is delivered from the second reservoir to the second channel; a first detection zone in the first channel disposed downstream of the fluid sample inlet and the first fluid inlet and a second detection zone in the second channel disposed downstream of the fluid sample inlet and the second fluid inlet; and means for determining a relative dispersivity of at least one molecule in fluid flowing through the first and second detection zones.

Another embodiment of the invention provides a microfluidic system. The system comprises a microfluidic device having a body structure including a first channel and a second channel formed therein; means for introducing a first fluid containing at least a first molecule into the first channel; means for introducing a second fluid containing at least a second molecule into the second channel; means for introducing a fluid containing one or more test molecules to both the first channel and the second channel; means for inducing pressure-driven flow of the first fluid, the second fluid, and the fluid containing the one or more test molecules in the first and second channels; means disposed in the first channel and the second channel for determining the dispersion of at least one of the first molecule, second molecule, or test molecule; and means for relating the dispersion to an interaction between two or more of the test molecule, the first molecule, and the second molecule.

The invention uses differences in diffusivities of molecules and the mitigating effect of the Taylor-Aris phenomenon on dispersion in molecular assays. A particular advantage of the invention is the ability to determine the interaction between molecules whose ratio of diffusivities is relatively small.

Also, the invention provides for assay detection methods that do not require labels that generate a discernible signal upon the occurrence of an associative or dissociative molecular interaction, or a separation step following a molecular association of labeled species.

The invention can be used to determine a variety of interactions between molecules, including associative and dissociative interactions. The methods, devices, and systems disclosed herein are particularly useful in measuring protein binding, such as universal protein binding assays for pharmaceutical libraries.

Further features and advantages of the present invention are described in detail below with reference to the accompanying drawings.

### BRIEF DESCRIPTION OF THE FIGURES

FIG. 1 shows an expected concentration profile versus channel axial position for a large molecule and a small molecule.

FIG. 2 shows a schematic representation of a fluid conduit system for practicing the present invention.

FIG. 3A shows a schematic representation of a microfluidic device for a single channel assay, including a pipettor element, a side channel, and a main channel.

FIG. 3B shows a schematic representation of an alternate microfluidic device for a self-referencing, single channel assay, including a pipettor element, a side channel, and a main channel.

FIG. 4 shows a schematic representation of a microfluidic device for a self-referencing, dual channel assay.

FIG. 5 shows a schematic representation of a microfluidic device for a single channel assay for use in a competitive binding experiment.

FIG. 6 shows a schematic of a microfluidic device for a single channel assay used in accordance with Example 1.

FIG. 7 shows the reference level of fluorescence from labeled biotin in accordance with Example 1.

FIG. 8 shows fluorescence signals obtained from repeated injections of labeled biotin in accordance with Example 1.

FIG. 9 shows normalized experimental fluorescence signal results of a binding assay experiment with labeled biotin and injections of buffer and Streptavidin in accordance with Example 1. The inset shows the results of a single injection.

FIG. 10 shows dispersion model results of a binding assay with biotin and Streptavidin in accordance with Example 1.

FIG. 11 shows a schematic representation of a microfluidic device for use in accordance with Example 2.

FIG. 12 is an illustration of an expected distribution of protein, ligand, and sample molecules in accordance with Example 2.

FIG. 13 shows the concentration of small and large molecules as a function of axial position in accordance with Example 2.

FIG. 14 is a further representation of the concentration of small and large molecules as a function of axial position in accordance with Example 2.

FIG. 15 is a further representation of the concentration of small and large molecules as a function of axial position in accordance with Example 2.

### DETAILED DESCRIPTION OF THE INVENTION

The invention provides novel methods, devices, and systems for determining interactions between a plurality of molecules using the Taylor-Aris dispersion phenomenon. Embodiments of the invention provide methods, devices, and systems using the Taylor-Aris dispersion phenomenon to determine interactions, including associative and dissociative interactions, between a plurality of molecules flowing in microfluidic conduits.

The invention incorporates the use of the Taylor-Aris dispersion phenomenon to detect, observe, measure, and analyze molecular interactions. The invention does not require tagged or labeled molecules for detection and is thus useful where such tags would interfere with the intermolecular interaction or where such labeling is not feasible. However, in some embodiments of the invention, labels or tags can be used.

In an embodiment, the invention has the advantage of microfluidic design and thus miniaturization, which allows small sample test sizes and conservative use of analytes. Similarly, the invention has the advantage of rapid sampling, which allows high-throughput and ready repetition of experimental results.

As discussed herein, “dispersion” is defined as convection-induced, longitudinal dispersion (sample broadening) of material within a fluid medium due to velocity variations across streamlines in laminar pressure-driven flow. For purposes of the invention, dispersion is generally defined as that due to the coupling between flow and molecular diffusion, i.e. Taylor-Aris dispersion. In this regime, the time-scale for dispersion due to convective transport is long or comparable to the time scale for molecular diffusion in the direction orthogonal to the flow direction. A detailed explanation of this phenomenon may be found in the Taylor & Aris papers mentioned above.

In a Taylor-Aris regime, the dispersion is characterized by rapid diffusion of molecules transverse to the pressure-driven flow along the axis of the conduit. Accordingly, molecules can “visit” both slow and fast regions of the flow field. Thus, when subjected to pressure-driven flow, on average a sample disperses more slowly as compared to a sample not under the Taylor-Aris regime. That is, the Taylor-Aris phenomenon mitigates dispersion of molecules of a fluid subjected to pressure-driven flow. See also U.S. Pat. No. 6,150,119 for its discussion of Taylor-Aris dispersion and its references cited therein, the patent incorporated by reference herein in its entirety.

The present invention utilizes differences in the diffusivities of molecules in determining interactions between the molecules. In particular, the inventors have discovered how to utilize the differences in diffusivity of large and small molecules in fluid flow in a conduit in the Taylor-Aris regime to determine the level of interaction between molecules.

The methods, systems, and devices of the invention are applicable for determining the interaction between molecules having a diffusivity ratio (diffusivity of a molecule with higher diffusivity/diffusivity of a molecule with lower diffusivity) of at least about 2. Illustratively, the ratio of diffusivities can be between 2 and 3, or even greater, such as, for example, about 8 to about 10. In other embodiments, the ratio of diffusivities may be between about 2 and about 10, or greater than about 10. A particular advantage of the invention is the ability to determine interactions between small and large molecules having a narrow ratio of diffusivity.

As is understood by one of ordinary skill in the art, the diffusivity of a molecule depends primarily upon its size. Typically, smaller molecules have higher diffusivities than larger molecules. For convenience, this application refers to “small” and “large” molecules as being representative of molecules having high and low diffusivities. However, it should be recognized that the diffusivity of molecules may depend upon other factors, including, but not limited to, the shape of the molecules.

The methods, systems, and devices of the present invention are particularly useful for determining interactions between small molecules and large molecules. The molecular weight of the small molecules can be about 5000 Da or less, for example about 300 Da to about 1000 Da. The molecular weight of the large molecules can be above about 15000 Da, or even significantly higher. It will be appreciated that it is not intended to limit the size of the molecules utilized in the present invention, so long as the molecules can be utilized in a conduit with flow conditions supporting the Taylor-Aris phenomenon, and have a diffusivity ratio of at least about 2. For example, if the molecules being tested are in the gaseous phase, the molecular weight for the small and/or large molecules can be less than those listed above.

The invention can be used in any fluid conduit where one can take advantage of the Taylor-Aris phenomenon, i.e., where the molecular diffusion across the conduit is on the order of or fast compared to the rate at which the molecules flow down the conduit. The conduit could be, for example, a covered channel in a microfluidic device or a capillary. As is understood by one skilled in the art, for a fixed velocity, the smaller the conduit, the more that the Taylor-Aris phenomenon mitigates dispersion due to pressure-driven flow. One may determine the optimum dimensions of the conduit to be used based upon, for example, the diffusivities of the molecules to be analyzed. See, e.g., U.S. patent application Ser. No. 10/206,787, filed Jul. 26, 2002, which is incorporated by reference herein in its entirety.

When analyzing interactions between molecules in liquid media, a suitable conduit can be a microchannel, or a conduit of even smaller cross-section. However, it should be understood that the conduit may be larger than a microchannel, provided that the Taylor-Aris phenomenon is present. The Taylor-Aris phenomenon could be present, for example, in an assay of molecules in the gaseous phase. Also, the shape of the conduit that can be used in the present invention is not particularly limited, and includes, for example, cylindrical, oval, and rectangular shaped conduits.

The types of molecules that may be utilized in embodiments of the inventive method include, but are not limited to, amino acids, polyamino acids, nucleotides, polynucleotides, saccharides, polysaccharides, antibodies, receptor proteins, signal proteins, enzymes, cofactors, cytokines, hormones, chemokines, polymers and drugs. It must be emphasized that this list is merely exemplary and that any of a variety of molecules can be used with the present invention. As used herein, the term analyte is meant to refer to these molecules when present in solution.

In an embodiment of the invention, the dispersion of at least one of a plurality of molecules flowing in a fluidic conduit (e.g. a large and a small molecule) is measured. The dispersion can be measured by a variety of methods known to those skilled in the art. In an embodiment, the dispersion is measured by detecting the concentration of one or more of the flowing molecules in the fluidic conduit.

Any means known to one of skill in the art may be used to detect the presence or concentration of the molecules within or arising out of the fluidic conduit. These means may include optical methods such as absorbance or fluorescence spectroscopy, thermal lens spectroscopy (see, e.g., Kitamori et al., Jpn. J. Appl. Phys. 39, 5316-5322, (2000)) and UV spectroscopy, electrochemical methods such as potentiometric and ampiometric detection, and other physical methods and chemical methods known to those skilled in the relevant art, including, but not limited to, mass spectroscopy, magnetic resonance techniques such as nuclear magnetic resonance or electron paramagnetic resonance, and radioactive measurement. Preferred means are by fluorescence or absorbance spectroscopy.

The present invention takes advantage of the knowledge that large molecules flowing in a fluid conduit do not laterally diffuse as rapidly as small molecules. As a result, when the large molecules are introduced into a conduit under pressure-driven flow, the dispersion of the large molecules by the range of flow velocities encountered across the conduit's cross-section is not reduced by lateral diffusion. This results in the large molecules being more prone to disperse while flowing through the conduit as compared to small molecules.

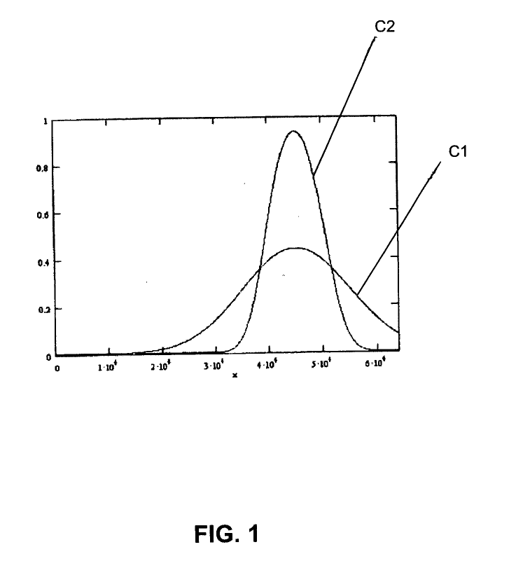

FIG. 1 illustrates the effect of the Taylor-Aris phenomenon on two identical pulses (i.e. pulses of the same concentration and duration) of large and small molecules introduced into a conduit. As the pulses flow down the conduit, the concentration profile of the large molecule pulse (as measured as a function of axial position in the conduit) will become broader as compared to the concentration profile of the small molecule because of the greater dispersivity of the larger molecule. FIG. 1 shows the concentration profile (C1) of a large molecule with diffusivity of 30 μ²/s and the concentration profile (C2) of a small molecule with diffusivity of 300 μ²/s as a function of axial position (x) in a fluidic conduit under pressure-driven flow at an arbitrary time after introduction of the large and small molecule pulses. As FIG. 1 illustrates, differences in the peak breadth and amplitude of the pulse concentration profiles develop as the pulses flow down the conduit. As can be seen, the profile (C2) of the smaller molecule pulse has a sharper, higher peak concentration as compared to the profile (C1) of the large molecule because the smaller molecule has diffused more rapidly across the fluid conduit (i.e. it has diffused in a direction transverse to the direction of flow), sampling different regions of the velocity profile, thus reducing the amount of dispersion.

The dispersion of the molecule or molecules flowing in the fluidic conduit is also related to the interaction between the plurality of molecules. For example, an interaction between the molecules occurs if the dispersion of at least one of the molecules is altered from the dispersion obtained in the absence of the other molecules.

The types of interactions that can be determined from the inventive method is not particularly limited. Illustratively, the interactions that may be determined by the present invention include associative interactions and dissociative interactions. Associative interactions include, but are not limited to, receptor/ligand interactions including antibody/antigen, complementary nucleic acids, nucleic acid associating proteins and their nucleic acid ligands; nucleic acid hybridization reactions, non-specific and specific binding, site-specific binding, catalytic protein recognition, receptor-substrate recognition, or enzyme/substrate, as well as other covalent (such as steric or electrostatic interaction), non-covalent, or ionic interactions between molecules. Dissociative interactions include, but are not limited to, the inverse of the associative reactions, as well as lysis or cleavage reactions where, for example, a relatively small labeled species is cleaved from a larger labeled substrate.

Of particular interest in practicing the present invention include interactions between biochemical molecules, such as, e.g., receptor-ligand interactions, enzyme-substrate interactions, cellular signaling pathways, transport reactions involving model barrier systems (e.g., cells or membrane fractions) for bioavailability screening, and a variety of other general systems.

For example, compounds may be screened for effects in blocking, slowing or otherwise inhibiting key events associated with biochemical systems whose effect is undesirable. For example, test compounds may be screened for their ability to block systems that are responsible, at least in part, for the onset of disease or for the occurrence of particular symptoms of diseases, including, e.g., hereditary diseases, cancer, bacterial or viral infections. Compounds that show promising results in these screening assay methods can then be subjected to further testing to identify effective pharmacological agents for the treatment of disease or symptoms of a disease.

Illustratively, the present invention can be used to screen for an effect of a test compound on an interaction between two components of a biochemical system, e.g., receptor-ligand interaction or an enzyme-substrate interaction. In this form, the biochemical system model will typically include the two normally interacting components of the system for which an effector is sought, e.g., the receptor and its ligand or the enzyme and its substrate.

Determining whether a test compound has an effect on this interaction then involves contacting the system with the test compound and assaying for the functioning of the system, e.g., receptor-ligand binding or substrate turnover. The assayed function is then compared to a control, e.g., the same reaction in the absence of the test compound or in the presence of a known effector.

The methods of the present invention may also be used to screen for effectors of much more complex systems where the result or end product of the system is known and assayable at some level, e.g., enzymatic pathways or cell signaling pathways. Alternatively, the methods and apparatuses described herein may be used to screen for compounds that interact with a single component of a system, e.g., compounds that specifically interact with a particular compound, such as a biochemical compound such as a receptor, ligand, enzyme, nucleic acid, or structural macromolecule. A more detailed discussion of biochemical interactions that may be assayed in the present invention is found in U.S. Pat. No. 5,942,443, which is incorporated by reference herein in its entirety.

As discussed above, the interaction of the plurality of molecules is typically accompanied by a detectable signal. For example, where the first molecule is a receptor and the second is a ligand, either the ligand or the receptor may bear a detectable signal. Although a labeled element may be used in embodiments of the invention, it should be emphasized that the present invention does not require the use of a labeled element. Thus, the invention is particularly useful where such labels or tags would interfere with binding or where such labeling is not feasible.

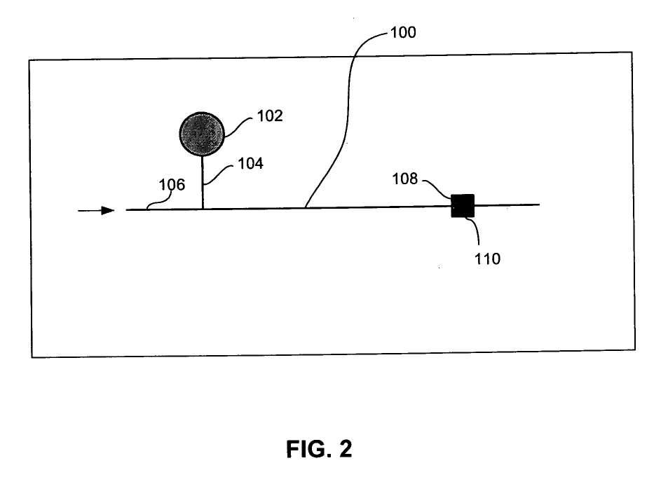

An apparatus in accordance with an embodiment of the invention is shown schematically in FIG. 2. A solution containing a large molecule can be introduced into conduit 100 from reservoir 102 via side channel 104 under pressure-driven flow conditions, operating in the Taylor-Aris regime. A discrete amount of solution containing the small molecule can then be introduced at point 106 in the conduit. In various embodiments, the solution containing the small molecule could be introduced from a reservoir (not shown), or from an external source via a pipettor (not shown). A detector (not shown) samples detection region 108 to detect the concentration of the small and/or large molecules at point 110.

As discussed above, under a Taylor-Aris regime the flowing small molecules will disperse less as they flow through the length of the conduit than will the flowing large molecules. If there were no interaction between the large and small molecules, the concentration of small molecules when detected would be expected to have a relatively sharp peak, because the rapid diffusion of the small molecules across the conduit mitigates dispersion due to pressure-driven flow. However, an interaction between the small and large molecules could modify the concentration profile of the small molecules. For example, if the small molecules were to bind to the large molecules, the resulting in a small molecule/large molecule complex that is larger than the small molecule. Consequently, the complex will disperse more rapidly than the small molecule. Accordingly, the resulting concentration profile for the bound smaller molecule would be shorter and broader than the concentration profile of the unbound smaller molecule. By analyzing the concentration profile of small molecules at detection region 108 after mixing with the large molecules, one can determine whether the small and large molecules have interacted.

The present invention provides yet another method for determining an interaction between a plurality of molecules. In methods in accordance with the invention, a first molecule of a plurality of molecules is introduced into a microfluidic conduit. A second molecule of the plurality of molecules is introduced into the microfluidic conduit. The dispersion of at least one of the molecules flowing in the microfluidic conduit is measured under pressure-driven flow conditions. The dispersion is then related to the interaction between the molecules.

The inventive microfluidic assay method incorporates the use of the Taylor-Aris dispersion phenomenon to detect, observe, measure and analyze molecular interactions which provides substantial benefits over previously described binding assay methods. The inventive method has the advantage of microfluidic design and thus miniaturization, which allows small sample test sizes and conservative use of analytes. Similarly, the inventive method has the advantage of rapid sampling, which allows high-throughput and ready repetition of experimental results.

As discussed above, the microfluidic assay method may be utilized to determine an interaction between molecules that have a ratio of diffusivities of at least about 2. In some embodiments, the ratio of diffusivities may be higher, such as between, e.g., about 2 and about 3, or greater, such as between about 2 and 10, or between about 8 and about 10, or even greater than 10.

In many cases, running the assay in the presence of a gel or other sieving matrix can increase the diffusvity ratio of two differently sized molecules. In general, a molecule traveling through a sieving matrix must negotiate a tortuous path defined by pores within the matrix. If the pore size of a sieving matrix is large compared to a particular molecule, then the diffusivity of that molecule will not be significantly affected by the presence of the matrix. On the other hand, the diffusivity of a molecule can be increased by as much as an order of magnitude if the molecule is large enough to have its movement impeded by the matrix. Thus, by employing an appropriate sieving medium in embodiments of the invention, the diffusivity ratio of a large molecule and small molecule can be increased. Sieving matrices that decrease the diffusivity of DNA, RNA, and protein molecules are commercially available in the form of gels. So, for example, a particular protein-ligand bonding pair that has a diffusivity ratio of 2 to 3 in solution might have a diffusivity ratio of 20 to 30 in a protein gel that decreases the diffusivity of the protein but does not significantly effect the diffusivity of the ligand. A sieving matrix such as a protein gel could fill all or a portion of conduit 100 in the embodiment of FIG. 2.

As used herein, the term “microscale” or “microfluidic” generally refers to structural elements or features of a device that have at least one fabricated dimension in the range of from about 0.1 micrometer to about 500 micrometers. When used to describe a fluidic element, such as a channel, passage, chamber, or conduit, the terms “microscale” or “microfluidic” generally refer to one or more fluid channels, passages, chambers or conduits which have at least one internal cross-sectional dimension, e.g., depth, width, length, or diameter, that is less than 500 micrometers, and typically between about 0.1 micrometer and about 500 micrometers. In an embodiment of the invention, the microscale channels, passages, chambers or conduits preferably have at least one cross-sectional dimension between about 0.1 micrometer and 200 micrometers. The microfluidic devices or systems used in accordance with the present invention typically include at least one microscale channel, usually at least two intersecting microscale channels, and often, three or more intersecting channels disposed within a single body structure. Channel intersections may exist in a number of formats, including cross intersections, “T” intersections, or any number of other structures whereby two or more channels are in fluid communication.

In many embodiments, the microfluidic devices will include an optical detection window disposed across one or more channels of the device. Optical detection windows are typically transparent such that they are capable of transmitting an optical signal from the channel over which they are disposed. For example, optical detection windows can be a region of a transparent cover layer, where the cover layer is glass or quartz or a transparent polymer material such as, for example, PMMA or polycarbonate. Alternatively, where opaque substrates are used in manufacturing the devices, transparent detection windows fabricated from the above materials may be separately manufactured into the microfluidic device. Suitable optical detection techniques include, but are not limited to, absorbance or fluorescence spectroscopy, thermal lens spectroscopy and UV spectroscopy.

However, in other embodiments, the detection system can include a non-optical detector or sensor for detecting a particular characteristic disposed within a detection region or zone. Suitable non-optical detection methods include, but are not limited to, electrochemical methods such as potentiometric and ampiometric detection and other physical methods and chemical methods known to those skilled in the relevant art, including mass spectroscopy, magnetic resonance techniques such as nuclear magnetic resonance or electron paramagnetic resonance, and radioactive measurement.

These microfluidic devices and the assay methods of the present invention may be used in a variety of applications which utilize the determination of associative and/or dissociative molecular interactions, such as in the performance of high-throughput screening assays in drug discovery, immunoassays, diagnostics, and nucleic acid analysis, including genetic analysis. As such, the devices used herein will often include multiple sample introduction ports or reservoirs for the parallel or serial introduction and analysis of multiple samples. Examples of such multiple sample introduction reservoirs is described in U.S. Pat. No. 5,976,336, which is herein incorporated by reference in its entirety. Alternatively, these microfluidic devices may be coupled to a multiple sample introduction port, e.g., a pipettor, which serially introduces multiple samples into the device for analysis. Examples of such sample introduction systems are described in U.S. Pat. Nos. 6,046,056 and 5,880,071, herein incorporated by reference in their entireties.

The present invention also provides assay methods in which microfluidic devices, systems and detection and analysis systems are used for generating and deconvoluting signal information such as the change in molecular dispersion in order to examine the interaction of molecules in solution. For example, the shape of the dispersion signal profiles for bound and unbound species in solution may be observed, measured and analyzed in order to quantitatively or qualitatively determine the extent to which one or more molecular analytes have interacted in the solution.

The reagents for carrying out the methods and assays of the present invention are optionally provided in kit form to facilitate the application of these assays for the user. Such kits may also include instructions for carrying out the subject assay, and may optionally include the fluid receptacle, e.g., the cuvette, multiwell plate, and microfluidic device, in which the assay is to be carried out.

Typically, reagents included within the kit include a label (if desired), as well as the microfluidic device and any necessary buffer solutions. The reagents may be provided in vials for measuring by the user, or in pre-measured vials or ampules that are simply combined to yield an appropriate mixture. The reagents may be provided in liquid and/or lyophilized form and may optionally include appropriate buffer solutions for dilution and/or rehydration of the reagents. Typically, all of the reagents and instructions are co-packaged in a single box or pouch that is ready for use.

In an embodiment of the invention, the methods involve the injection and flow of pulses of sample materials (“slugs”) through a microscale fluidic conduit, whereby a reagent introduced into the conduit through a side channel causes a molecular interaction to occur such as, e.g., an associative or dissociative interaction. The conduit may exist as a discrete conduit, e.g. a capillary or tube into which the reagent and sample materials are introduced, or as a channel in an integrated microscale channel network or microfluidic device in which various steps, including the sampling of one or more components and/or the mixing of the different components of the mixture takes place.

In an embodiment of the invention, a first molecule can be introduced into the microfluidic conduit in a continuous stream of fluid, and a second molecule can be introduced into the microfluidic conduit in a bolus of fluid so that the first and second molecules are in fluid communication.

Sample slugs subjected to pressure-driven flow in microfluidic conduits spread via Taylor-Aris dispersion, in which the dispersivity is inversely proportional to the molecular diffusivity. Computer-controlled pressure may be used to gain precise control over fluid motion in the microfluidic channel network. A suitable pressure control system is described in U.S. patent application Publication No. 2001/0052460, which is incorporated by reference herein in its entirety. Although the benefits realized by the present invention are primarily due to Taylor-Aris dispersion occurring in pressure-driven flow, electrokinetic or electroosmotic forces may be additionally utilized so long as they do not unduly interfere with the Taylor-Aris regime.

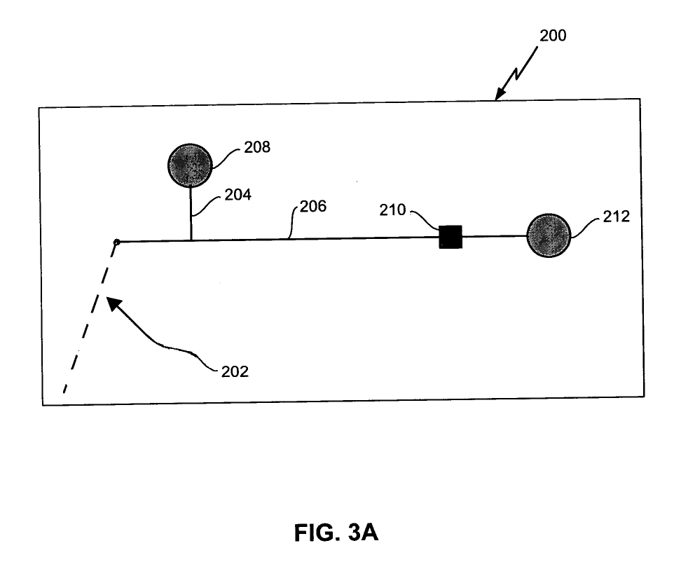

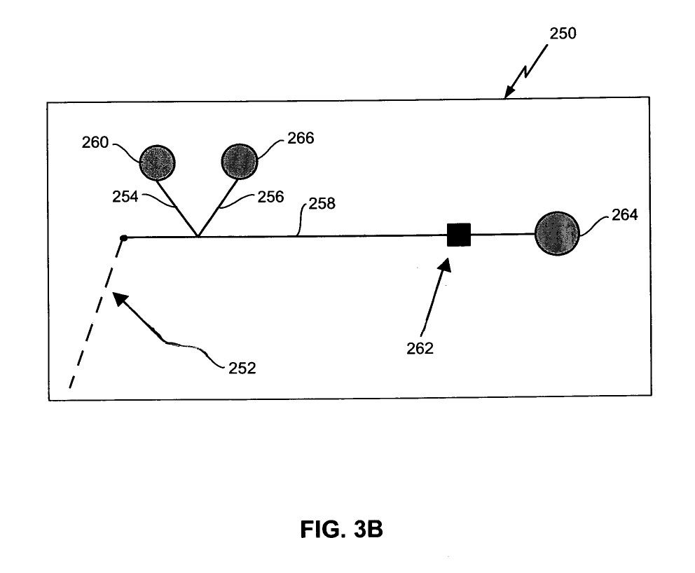

In an embodiment, the invention comprises a single channel microfluidic molecular binding assay. An example of a suitable microfluidic device with a single channel configuration for use with this embodiment of the invention is illustrated in FIG. 3A. FIG. 3A shows a microfluidic device comprising a planar substrate into which grooves that form channels 204 and 206 have been etched. A transparent cover plate overlies the planar substrate. The cover plate comprises two apertures that form reservoirs 208 and 212 respectively. The microfluidic device 200 also includes a pipettor element or a sampling element such as a capillary glass tube (“sipper”) 202 that protrudes downward from the planar substrate, and intersects channel 206 at intersection 205. In this particular design, a solution containing a small molecule is drawn into sipper 202 and then into main channel 206, while a solution containing a large molecule, such as a protein solution, flows from reservoir 208, via side channel 204, in a steady manner into the main channel 206. “Single channel” refers to the single main channel 206 in which the dispersion of the molecules is measured.

As discussed above, a variety of detection methods can be used to detect the concentration of one or more molecules. In the embodiment of FIG. 3A, a steady level of absorbance (or fluorescence, or other parameter, depending on the detection method) can be observed in detection region 210 from the molecules flowing into the main channel. When a slug of small molecule is brought up through sipper 202, the sample slug will be brought into contact with the large molecule (protein) stream, entering via side channel 204, and thoroughly mixed.

In an embodiment, the dispersion of the molecules is compared to the dispersion of the first molecule flowing in the microfluidic conduit in the absence of the second molecule. Alternatively or additionally, the dispersion of the molecules is compared to the dispersion of the second molecule flowing in the microfluidic conduit in the absence of the first molecule.

For example, if the small molecule discussed above in relation to FIG. 3A binds to the protein, the bound small molecule will disperse more in the fluid stream as compared to smaller molecules in the absence of binding. However, if there is no affinity between the small molecule and the large protein molecule, the concentration profile (and dispersion) of the small molecule will not change. Based on the detected concentration peak shape (width or height), it is therefore possible to detect, observe, measure and analyze a binding event. Once past detection region 210, the fluid mixture terminates at a waste reservoir 212.

In some embodiments consistent with the device in FIG. 3A, the sipper 202 sequentially samples a series of test compounds to determine whether each test compound binds to the protein introduced into main channel 206 from reservoir 208. In a variation of the embodiment of FIG. 3A, the protein and a test compound are mixed off the microfluidic device, and the resulting mixture is introduced into the microfluidic device via sipper 202. Since this variation does not require that protein solution be introduced from reservoir 208, the design of microfluidic device 200 could be simplified by eliminating reservoir 208 and channel 204. Fluid flow through the simplified device could be controlled by means of a single pressure source, such a vacuum source coupled to waste reservoir 212.

FIG. 3B illustrates an alternate embodiment of a microfluidic device using a single channel, with multiport control. The chip design 250 includes a sipper 252, side channels 254 and 256, and a main channel 258. In this design, a solution containing small molecules is drawn into sipper 252 and then into main channel 258 while a larger molecule, such as a protein solution, flows under pressure from a protein reservoir 260, via side channel 254, in a steady manner into the main channel 258, to detection region 262 for quantitation, and to waste reservoir 264. The resulting concentration profile is determined. Next, the flow from the protein reservoir 260 is turned off, and a solution containing the small molecules is drawn into the main channel 258 with a buffer solution flowing from reservoir 266 via side channel 256, into the main channel 258, to detection region 262, and to waste 264.

The concentration profiles for the small molecule mixing with the protein and with the buffer are compared. If there is an interaction between the small molecule and the protein, such as binding of the small molecule to the protein, the concentration profile will have a shorter and broader peak as compared to that of the small molecule/buffer stream. However, if the peak amplitude and width are equivalent for the two streams, then no binding event has occurred.

FIGS. 3A and 3B merely represent embodiments within the scope of the invention of a microfluidic device using a single channel and are not meant to limit the intended scope of the invention. It should be recognized by one of ordinary skill that a large number of potential chip designs would be operable to perform in accordance with the invention.

In another embodiment, the invention includes a microfluidic system comprising a microfluidic device with a dual channel design. The device can include a body structure having first and second channels formed therein that may intersect each other. The system also includes a fluid sample inlet through which a sample is delivered to the first channel and the second channel. The system may also include fluid reservoirs in fluid communication with the first and second channel, through which fluids may be delivered from the reservoirs to the channels. Further, the system may include detection zones in the first and second channels. The detection zones may be disposed downstream of the fluid sample inlet and the inlets to the channels in fluid communication with the fluid reservoirs. The system may also include means for determining a relative dispersivity of at least one molecule in fluid flowing through the first and second detection zones.

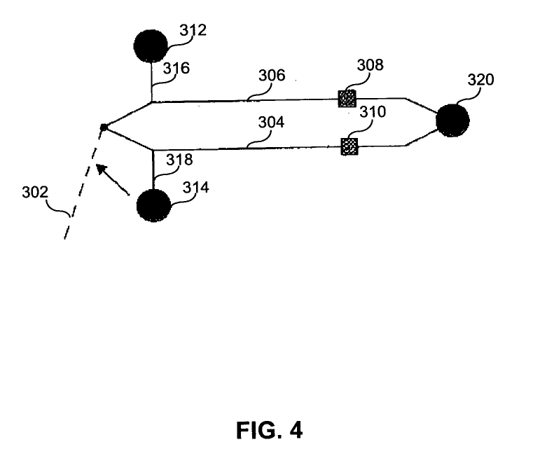

FIG. 4 illustrates an example of such a system and its use. A slug of solution containing a small molecule is drawn into a sipper 302 and the slug is split into two channels, reference channel 304 and test channel 306. After the split, there exist two fluidically equivalent circuits. Half of the slug is mixed in test channel 306 with a protein solution from protein reservoir 312 and sent to detection region 308 for quantitation. The other half of the slug is mixed in reference channel 304 with buffer solution from buffer reservoir 314 and sent to detection region 310. Protein and buffer are introduced via side channels 316 and 318, respectively. After passing detection regions 308 and 310, the channels both lead to waste reservoir 320. In the embodiment of FIG. 4, both channels 304 and 306 are in direct fluid communication with waste reservoir 320. In alternative embodiments, the two channels could merge, and the resulting single channel would lead to waste reservoir 320.

As discussed above for FIGS. 3A and 3B, if the peak amplitude and width of the concentration profile of the small molecule are equivalent for the two streams, then one can conclude that no binding event has occurred. However, if there is significant broadening of the small molecule concentration profile for the stream that interacts with the protein, then one can conclude a binding event has occurred.

Also, in embodiments of the invention, one or more additional molecules can be introduced into the microfluidic conduit, and the dispersion of the molecules flowing in the conduit can be measured. Illustratively, the present invention encompasses a competitive binding assay using different potential binding analytes. In such an assay, the measurement of dispersion is used to determine the extent to which each competing molecule binds to another (typically a larger) molecule.

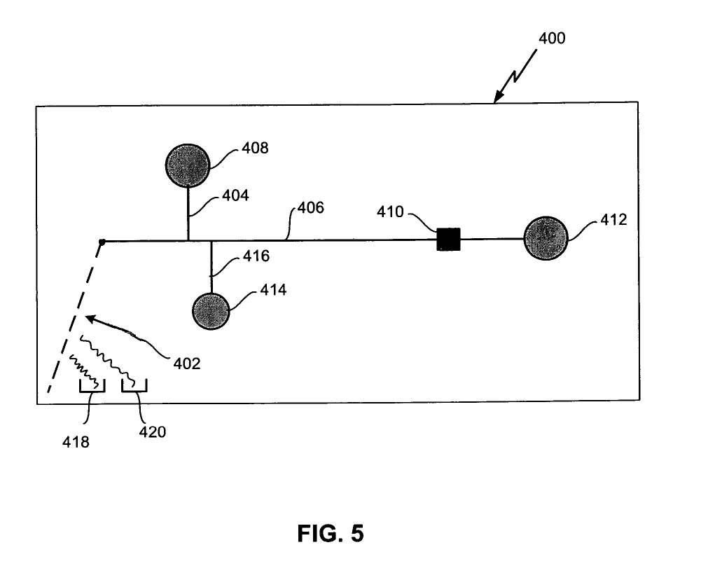

FIG. 5 illustrates an example of a competitive binding assay. Buffer solution is sipped from a container 418 by sipper 402 so that it fills the fluidic network and flows through main conduit 406 at a steady rate. Protein solution from reservoir 408 is also introduced into main conduit 406 at a steady rate via side channel 404. Protein and buffer solution flow through main conduit 406 past detection region 410 to waste 412. Using pressure control, discrete slugs of fluorescently labeled ligand are introduced into main conduit 406 from reservoir 414 via side channel 416. The concentration of the ligand introduced into the main conduit is measured at detection region 410 and a concentration profile for the ligand is determined.

Next, the sipper 402 samples solution from a second container 420. The solution in the second container 420 contains a small molecule (a test compound). This solution is introduced at a steady rate from container 420 by sipper 402 and flows through main conduit 406 with protein solution flowing from reservoir 408 via side channel 404. With the protein and small molecule solutions flowing, discrete slugs of the fluorescently labeled ligand are pulsed under pressure control into main conduit 406 from reservoir 414 via side channel 416. The concentration of labeled ligand flowing through the main conduit is measured at detection region 410 and a concentration profile for the ligand is determined.

When a test compound has an effect on the interaction of the protein with the ligand, a variation will appear in the signal produced by the detected ligand. For example, if a test compound inhibits the interaction of the ligand with the protein, e.g. inhibits binding of the ligand to the protein, the unbound ligand will continue to behave as a small molecule, rapidly sampling different portions of the pressure-driven velocity profile, which would result in reduced dispersion and a sharper peak in its concentration profile (measured by the fluorescence signal). On the other hand, if the test molecule enhances the interaction of the ligand with the protein, e.g. increases binding of the ligand to the protein, the bound ligand will diffuse across the conduit more slowly, and result in greater dispersion and a broader and shorter peak in its concentration profile. If the test molecule does not affect the interaction of the ligand and protein, the concentration profile of the detected ligand will not change from the absence of the test molecule. After obtaining a sample from the second container 420, the sipper 402 can obtain samples from other containers. In some embodiments, the containers are wells in a multiwell plate.

Embodiments of the invention have been described above that include separate introduction of a plurality of molecules. However, it should be noted that the order of or manner of introduction of the plurality of molecules is not particularly limited. For example, the plurality of molecules can be introduced simultaneously. Illustratively, a plurality of molecules can be pre-mixed and introduced in a bolus of fluid. For example, in a variation of the competitive assay of FIG. 5, solutions comprising the protein, the ligand, and various test compounds could be prepared off of the microfluidic device. Slugs of these solutions could then be serially introduced into the microfluidic device through a sipper such as sipper 402 in the embodiment of FIG. 5. Just as in the embodiment of FIG. 5, the concentration profile of the labeled ligand in each slug would be determined by measuring the concentration of the ligand as it passes through detection region 410. As previously described, comparing the shape of the concentration profile produced in the presence of a test compound to the concentration profile produced in the absence of any test compound indicates whether the test compound affects the interaction between the ligand and the protein.

One skilled in the art will readily recognize that additional embodiments comprising the use of the Taylor-Aris dispersion phenomenon and a plurality of reference, test, sipper and other conduits as well as detectors are clearly within the scope of the invention. The invention is intended to encompass any method using the Taylor-Aris phenomenon in conduits to determine molecular interactions.

### EXAMPLES

The following examples are provided to further illustrate the present invention. It is to be understood, however, that these examples are for purposes of illustration only and are not intended as a definition of the limits of the invention.

### Example 1

### Protein Binding Assay

Microfluidic Device Design & Instrumentation:

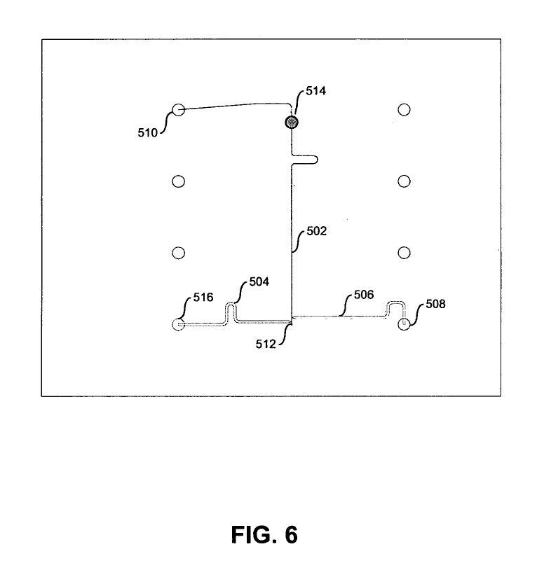

The microfluidic device design used was a SP299A single sipper chip (Caliper Technologies Corp., Mountain View, Calif.). The microfluidic circuit of the SP299A device is shown in FIG. 6 and consists of a sipping capillary (“sipper”) (not shown) in fluidic connection with a main channel 502 and two side channels 504 and 506. The sipper is physically attached to the chip at point 512.

The instrument used for this experiment was a Caliper 100 single sipper system (Caliper Technologies Corp., Mountain View, Calif.) (not shown). The instrument included an x-y-z robot that was used to present the microtiter plate to the sipper for sampling reagents stored in the microtiter plates.

In addition, fluorescent optics (i.e. light source, photodiode, detection/collection lenses, filters etc.) were used to detect samples in the main channel of the device. For this set of experiments, the excitation/emission filter set was 485 nn/535 nm. Additional hardware on the Caliper 100 system included a syringe pump for applying a driving vacuum at well 510 of the device. The instrument was controlled and the data collected and analyzed by a computer connected to the instrument.

The device was operated under a steady vacuum applied at a well 510. The hydrodynamic resistances of the fluidic circuit were designed such that 70% of the flow delivered to main channel 502 originates from the sipper, with 15% coming from each of side channels 504 and 506.

Samples were brought onto the device via the sipper by placing them in microtiter plates, and these samples were reacted with reagents present on the device that are delivered to main channel 502 via two side channels 504 and 506. The side channels were in fluidic connection with reagent wells into which fluids were dispensed using a standard pipettor up to a maximum capacity of approximately 40 microliters.

The flow rates at a driving pressure of −1 psi applied at well 510 were 0.56 nl/s in the sipper and 0.11 nl/s from each of the side channels for a total flow rate in the main channel of 0.78 nl/s. The transit time from the distal end of the sipper to intersection 512 with main channel 502 was approximately 11.3 seconds, while the transit time from intersection 512 to detection region 514 was approximately 33.7 seconds, for a total transit time from sipper to detection region 514 of approximately 45 seconds.

Reagents:

The following reagents were used in the experiment: assay buffer (50 mM HEPES, pH 7.5); labeled biotin, with a T₁₀ linker to prevent fluorescent quenching upon binding with Streptavidin (fluorescein attached to biotin by a linker of 10 thymidine residues), custom synthesized from Oligos Etc., Inc. with molecular weight of 3100 g/mol; and Streptavidin (Sigma, product no. S4762), having molecular weight of approximately 60,000 g/mol.

Obtaining the Unbound Reference Signal:

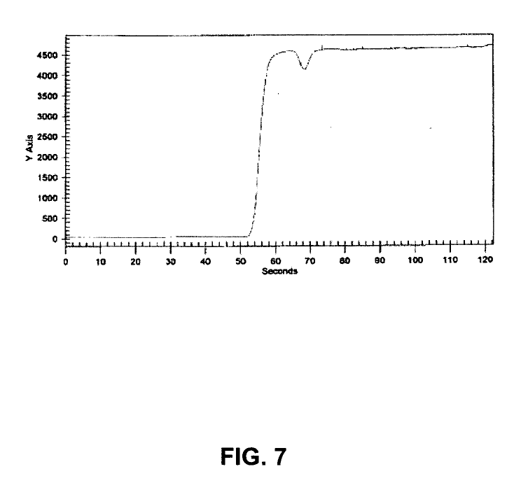

In order to ascertain the background or baseline fluorescence conditions, a first run was conducted using injections with buffer in side channels 504 and 506. For these first set of experiments, 50 mM HEPES was loaded into wells 508 and 516, while alternately sipping 50 mM HEPES (buffer) and 5 μM Bi-T₁₀-Fl (sample) from wells of a microtiter plate (not shown). Prior to sending a pulsed series of sample injections into the device via the sipper, a reference level of fluorescence was taken by continuously sipping a Bi-T₁₀-Fl sample until a steady signal was achieved, as shown in FIG. 7, which illustrates the fluorescence signal on the y-axis verus time. The reference level of fluorescence was used to normalize the injection data.

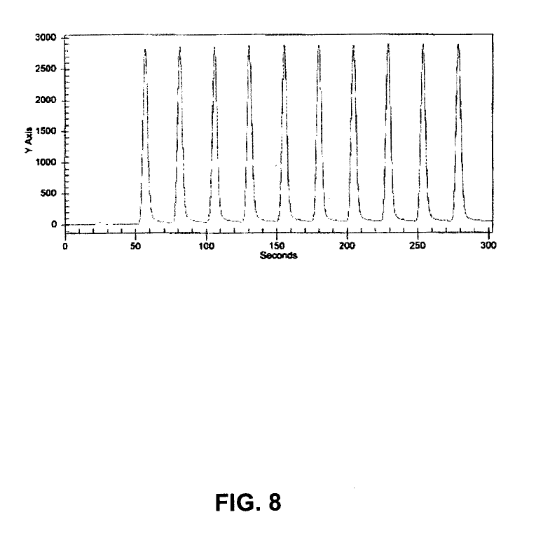

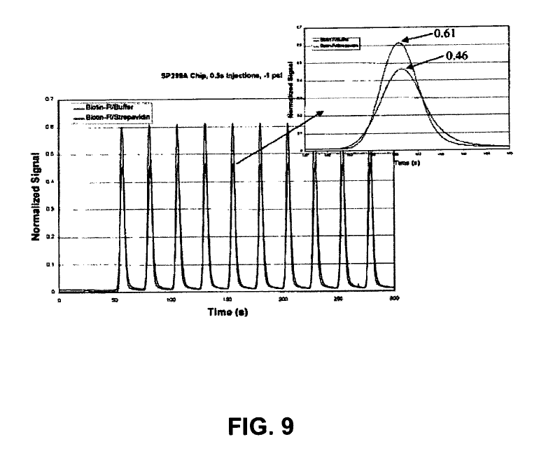

Next, the instrument was programmed to perform a series of buffer-sample sip cycles, in which the dwell times in the wells were 20 seconds and 0.5 second respectively. The fluorescence levels measured are shown in FIG. 8. The sample injections did not achieve the same level of fluorescence as the reference level, due to dispersion of the injected samples, resulting in a reduction in the observed concentration at detection region 514 (and a corresponding broadening of the peak relative to the injection time). The raw injection data in FIG. 8 normalized relative to the reference shown in FIG. 7 are shown in FIG. 9 (Biotin-Fl/Buffer, taller peaks). The data was normalized using relative to the reference using the following relationship:

$$NORMALIZED SIGNAL = \frac{RAW SIGNAL}{( REFERENCE MAX - REFERENCE MIN )}$$

Binding assay with Injections of Streptavidin:

Referring again to FIG. 6, 10 μM Streptavidin in assay buffer was placed in wells 508 and 516 instead of the HEPES buffer, and a run was conducted with Streptavidin. The flow conditions were identical to those discussed above for the alternating buffer/sample sip cycles.

As the total flow rate from side channels 504 and 506 was 30% of the total flow rate in main channel 502, the concentration of Streptavidin in the main channel was 3 μM (10 μM×0.30). As each Streptavidin has 4 binding sites per molecule, the concentration of binding sites was 12 μM (4×3 μM). As the sipper contributes 70% of the total flow rate to the main channel, the concentration of Bi-T₁₀-Fl in the main channel downstream of the side channels was 3.5 μM (0.7×5 μM). Thus, there were an excess of approximately 3.4-fold (12/3.5) binding sites versus binding molecules in this experiment, to ensure that all of the biotin would be bound just downstream of side channels 504 and 506. Reference and injection data (not shown) were acquired as described above.

The normalized results of the Bi-T₁₀-Fl/Streptavidin binding assay are shown in FIG. 9, plotted along with the experimental results with buffer in side channels 504 and 506. The inset in FIG. 9 shows a zoomed view of a single injection.

As is shown in FIG. 9, the peak signals for the injected Bi-T₁₀-Fl interacting with Streptavidin are shorter and broader as compared to the Bi-T₁₀-Fl/buffer assay, indicating that the bound biotin species showed enhanced dispersion relative to the unbound species. The normalized peak maxima for the bound/unbound cases were 0.61/0.46 respectively. The data indicate the increased dispersion of the biotin when Streptavidin is in the side channels, illustrating that a determination of an interaction between a plurality of molecules (here, biotin and Streptavidin binding) can be made by analysis of the level of dispersion of the bound and unbound molecules.

Model Results:

The mathematics of diffusion and dispersion are well understood as applied to microchannels. The experimental results were compared to a mathematical model with the following assumptions:

(1) Bi-T₁₀-Fl diffusion coefficient: 236 μm²/s (estimated based on molecular weight);

(2) Streptavidin (or bound complex) diffusion coefficient: 81 μm²/s (estimated based on molecular weight); and

(3) Initial sample injection slug size based on the flow rate and injection time for the experiments.

The mathematical formula that governs the dispersion of a slug of fluid appearing as square pulse is given by:

$$C ( x , t ) = 0.5 \cdot erf ( \frac{\frac{h}{2} - x}{\sqrt{4 \cdot {K}_{eff} \cdot t}} ) + erf ( \frac{\frac{h}{2} + x}{\sqrt{4 \cdot {K}_{eff} \cdot t}} )$$

where C(x,t) is the concentration at point x at time t, h is the initial length of the concentration slug, and K_{eff} is the effective dispersivity coefficient, which is a function of the molecular diffusivity, microchannel geometry, and linear fluid velocity.

Details of the derivation of the above equation can be found in the works of Sir Geoffrey Taylor and R. Aris in the papers referred to above, which are incorporated herein by reference. The model can account for dispersion that occurs in the capillary prior to the side channels in addition to the dispersion that occurs downstream of the side channels for both the unbound and bound cases.

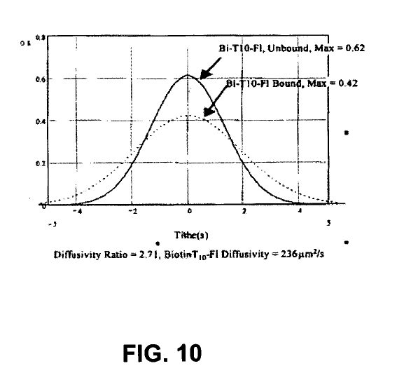

Dispersion model results are shown in FIG. 10 for the bound and unbound species. The time axis should be interpreted as an elapsed time, in which the center of the peak is centered at t=0. As can be seen from FIG. 10, the bound peak is both shorter and broader, indicating that it is more disperse. The model results are in quantitative agreement with the experimental data shown in FIG. 9.

### Example 2

Off-Chip Competitive Binding Assay

This example summarizes how one would implement an off-chip competitive binding assay based on differential Taylor-Aris dispersion of bound and unbound molecules.

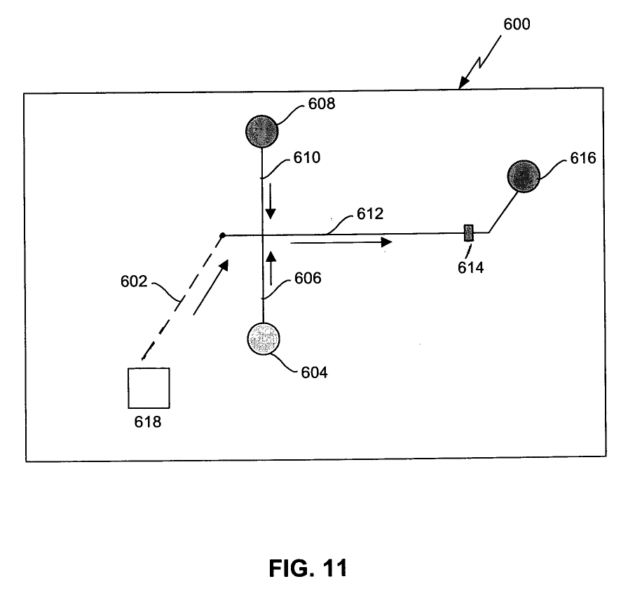

To perform such an assay, one could use a chip design as illustrated in FIG. 11. Chip 600 includes a sipper 602, two side channels 606 and 610, a main channel 612, reservoirs 604 and 608, detection region 614, and waste 616. External to the chip is a microtiter plate well 618.

As is illustrated in FIG. 11, both protein and ligand are delivered continuously to main channel 612 from reservoirs 604 and 608 and side channels 606 and 610, respectively, while a small molecule that is being assayed for competitive binding to the protein is drawn up from a microtiter plate well 618 through the sipper 602 in slugs that are separated by buffer spacers. The ligand is known to bind to the protein of interest, and is fluorescently labeled.

As the ligand is the only fluorescent species in the chip, the signal observed at detection region 614 can be determined by examining the fluorescent signal emanating from the ligand species (or any complexes involving the ligand) during the course of the experiment. All other species, i.e. the small molecule, the protein, or complexes of the small molecule and protein do not contribute to the fluorescent signal.

Since the flow fraction supplied by the ligand side channel does not change over time, one would expect a steady level of fluorescence observed at the detection region. Nevertheless, differential dispersion can redistribute the amount of ligand in the channel, and provide a data signature that is indicative of a change in the degree of binding between the protein and ligand.

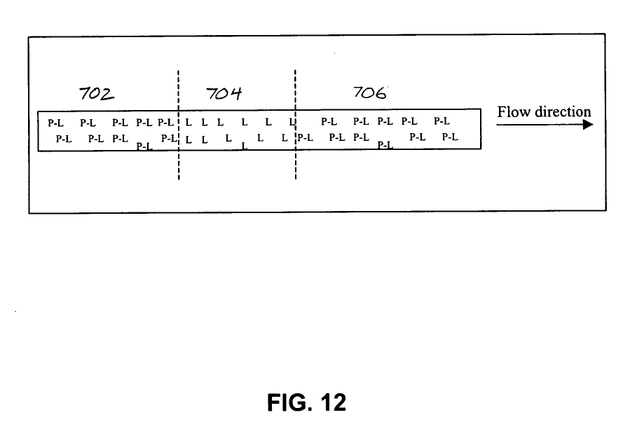

FIG. 12 represents the distribution of ligand (L) and protein-ligand complex (P-L) in a region of channel 612 just downstream of where side channels 606,610 intersect channel 612 that results from the injection of a slug of a molecule that prevents the ligand from binding with the protein. The leading portion 706 of the fluid flowing through channel 612 represents a portion of the fluid into which the sipper introduced a buffer spacer. In portion 706 there is nothing preventing the ligand from binding to the protein, so portion 706 contains protein ligand complex. In portion 704, however, the sipper introduced a slug of a molecule that competes with the ligand for binding sites on the protein. In the example embodiment of FIG. 12, the protein essentially completely binds with the introduced molecule to the exclusion of the ligand. Since in portion 704 the ligand does not bind with protein, the ligand in portion 704 is unbound. Trailing portion 702 represents a second portion of the flow into which the sipper introduced a buffer spacer. As was the case in portion 706, the ligand in portion 702 is part of a protein-ligand complex.

Assuming that the quantum efficiency of the protein-ligand complex is equivalent to that of the ligand, it would seem that a change in signal would not be observable, as the total number of ligand molecules (either bound or unbound) remains fixed. This is not the case, however, because differential Taylor-Aris dispersion will occur at the interface between a solution containing unbound ligand and a solution containing protein-ligand complex due to the difference in diffusivity for the two species. The unbound ligand will have a higher diffusivity because of its smaller size. Consequently, the larger protein-ligand complex will disperses more quickly.

One can use a mathematical model to investigate the expected data signature. The initial condition is similar to the schematic shown in FIG. 12, in which a slug of the small molecule (unbound ligand) is bounded by regions containing the big molecule (protein-ligand complex).

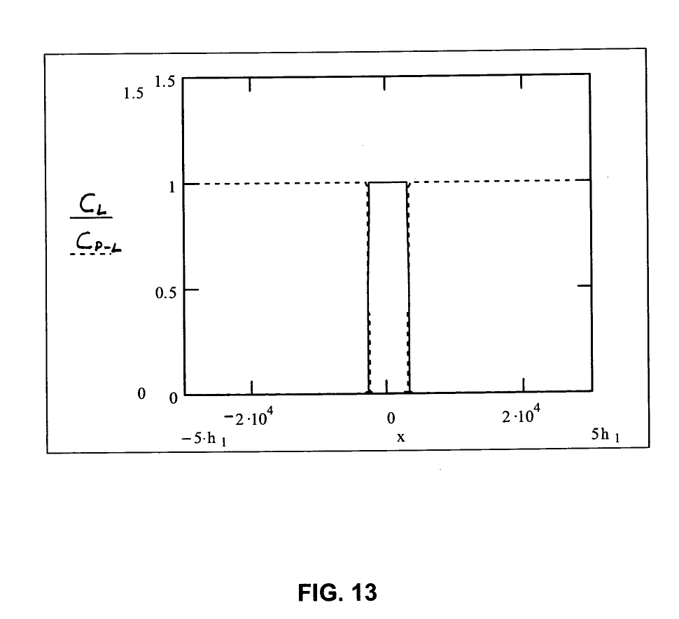

FIG. 13 illustrates the concentration of the small molecule and adjacent big molecule as a function of the channel axial position for a short time (i.e., just downstream of the side channels). In other words, the concentration profile in FIG. 13 corresponds to the situation shown in FIG. 12. The solid line represents the concentration profile of the unbound ligand, while the dotted line represents the concentration of ligand present in the protein-ligand complex. In FIG. 13 the overall concentration of ligand, which is the sum of the concentration of bound and unbound ligand, is the same at all points in channel. This constant concentration has been set to an arbitrary value of 1 concentration unit. Since in this embodiment the quantum efficiency of the bound ligand in the protein-ligand complex is equivalent to that of the unbound ligand, the fluorescent signal emanating from the portion of the channel represented in FIG. 13 would be constant.

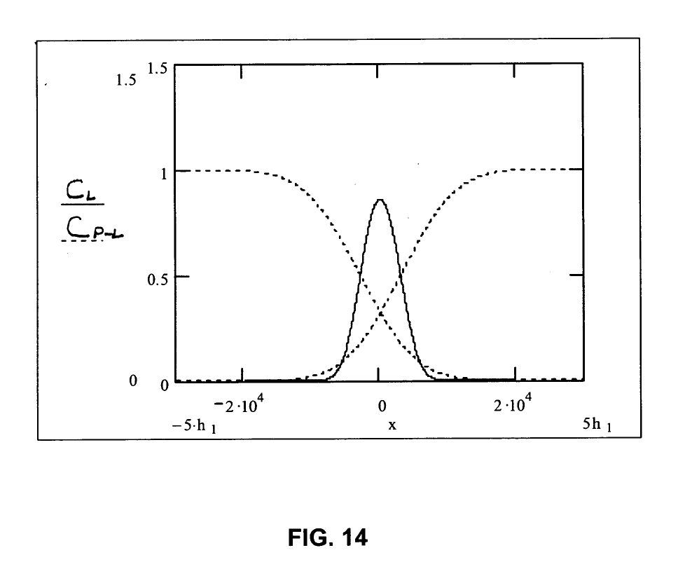

FIG. 14 illustrates the concentration profile for the same portion of fluid after it has flowed to a point in the channel downstream of the point represented in FIG. 13. The calculated concentration profile in FIG. 14 is based on the assumption that the small molecule diffuses 10 times faster than the large molecule. Since the fluorescence emanating from bound and unbound fluorescently labeled bound and unbound ligand is identical, the overall fluorescent signal at the detection region will be the sum of the two.

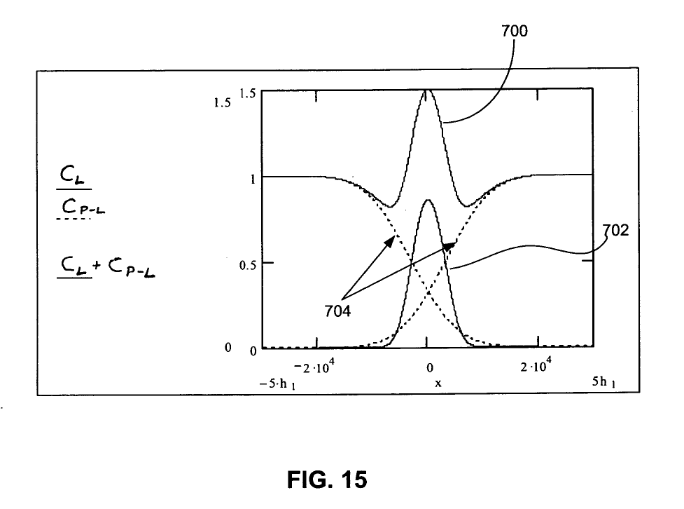

The overall fluorescence emanating from FIG. 14 is shown in FIG. 15. In FIG. 15, curve 700 is the observable signal at the detection region, and is the sum of the signal from the ligand (curve 702) and the ligand-protein complex (curve 704). The deviation in the signal from the initial value of 1 is caused by differential dispersion. In this embodiment, the data signature is a dip, followed by a peak, followed by a dip. Generally, the peak will be larger than the dip, as this originates from the species that disperses more slowly. The magnitude of the data signature (from the dip to the peak), should be proportional to the degree of binding, and could be used for quantification.

It is noted that the teachings herein can be extended to any application where different chemical interactions are determined at different locations in a flowing system.

It will be apparent to those skilled in the relevant art that the disclosed invention may be modified in numerous ways and may assume embodiments other than the preferred form specifically set out and described above. Accordingly, it is intended by the appended claims to cover all modifications of the invention that fall within the true spirit and scope of the invention.

What is claimed is:

1. A method for determining an interaction between a plurality of molecules, comprising:
(a) flowing a plurality of molecules in a fluidic conduit;
(b) measuring the dispersion of at least one of the molecules, wherein the dispersion of the molecules is Taylor-Aris dispersion, and
(c) relating the dispersion to the interaction between the plurality of molecules.

2. The method of claim 1, wherein the molecules are subjected to pressure-driven flow.

3. The method of claim 1, wherein the dispersion of the at least one molecule is compared with the dispersion of each of the plurality of molecules in the absence of the other molecules.

4. The method of claim 1, wherein the dispersion is measured by detecting the concentration of the molecules in the fluidic conduit.

5. The method of claim 4, wherein the molecules are not labeled for detection.

6. The method of claim 1, wherein the interaction is an associative interaction.

7. The method of claim 6, wherein the molecules comprise a ligand and a receptor.

8. The method of claim 1, wherein the interaction is a dissociative interaction.

9. The method of claim 1, wherein the plurality of molecules are selected from the group consisting of amino acids, polyamino acids, nucleotides, polynucleotides, saccharides, polysaccharides, antibodies, receptor proteins, signal proteins, enzymes, cofactors, cytokines, hormones, chemokines, polymers and drugs.

10. The method of claim 1, wherein a diffusivity ratio of two of the molecules is at least about 2.

11. A method for determining an interaction between a plurality of molecules, comprising:
(a) introducing a first molecule of a plurality of molecules into a microfluidic conduit;
(b) introducing a second molecule of the plurality of molecules into the microfluidic conduit;
(c) measuring the dispersion of at least one of the first and second molecules flowing in the microfluidic conduit under pressure-driven flow conditions; and
(d) relating the dispersion to the interaction between the plurality of molecules.

12. The method of claim 11, wherein the first molecule is introduced into the microfluidic conduit in a continuous stream of fluid.

13. The method of claim 11, wherein the second molecule is introduced into the microfluidic conduit in a bolus of fluid.

14. The method of claim 11, wherein the first and second molecules are introduced simultaneously.

15. The method of claim 14, wherein the first and second molecules are pre- mixed and introduced as a bolus of fluid.

16. The method of claim 11, wherein the first and second molecules are in fluid communication.

17. The method of claim 11, wherein the dispersion is measured by detecting the concentration of the molecules.

18. The method of claim 17, wherein the detection is by fluorescence, absorbance spectroscopy, thermal lens spectroscopy, or UV spectroscopy.

19. The method of claim 17, wherein the detection is by mass spectroscopy, an electrochemical technique, a magnetic resonance technique, or radioactive technique.

20. The method of claim 11, wherein the dispersion of the first molecule is compared to the dispersion of the first molecule flowing in the microfluidic conduit in the absence of the second molecule.

21. The method of claim 11, wherein the dispersion of the second molecule is compared to the dispersion of the second molecule flowing in the microfluidic conduit in the absence of the first molecule.

22. The method of claim 11, wherein a diffusivity ratio of two of the plurality of molecules is at least about 2.

23. The method of claim 22, wherein the diffusivity ratio is about 8-10.

24. The method of claim 22, wherein the diffusivity ratio is greater than 10.

25. The method of claim 11, wherein the interaction is an associative interaction.

26. The method of claim 11, wherein the interaction is a dissociative interaction.

27. The method of claim 11, wherein the plurality of molecules are selected from the group consisting of amino acids, polyamino acids, nucleotides, polynucleotides, saccharides, polysaccharides, antibodies, receptor proteins, signal proteins, enzymes, cofactors, cytokines, hormones, chemokines, polymers and drugs.

28. The method of claim 11, wherein the molecules comprise a receptor and a ligand.

29. The method of claim 11, wherein the molecules comprise an enzyme and a substrate.

30. The method of claim 11, further comprising introducing one or more additional molecules into the microfluidic conduit, and measuring the dispersion of the one or more additional molecules flowing in the conduit.

31. The method of claim 11, wherein measuring the dispersion comprises measuring longitudinal dispersion in the axis of flow.

32. The method of claim 11, wherein the first and second molecules do not flow in side-by-side streams.

33. A microfluidic system, comprising:
a microfluidic device having a body structure including a first channel and a second channel formed therein, wherein the first and second channels intersect;
a fluid sample inlet through which a sample is delivered to the first channel and the second channel;
a first fluid reservoir in fluid communication with the first channel, the first channel having an inlet through which a first fluid is delivered from the first reservoir to the first channel;
a second fluid reservoir in fluid communication with the second channel, the second channel having an inlet through which a second fluid is delivered from the second reservoir to the second channel;
a first detection zone in the first channel disposed downstream of the fluid sample inlet and the first fluid inlet and a second detection zone in the second channel disposed downstream of the fluid sample inlet and the second fluid inlet; and
means for determining a relative dispersivity of at least one molecule in fluid flowing through the first and second detection zones.

34. The system of claim 33, wherein the sample inlet is coupled to a source of a sample fluid.

35. The system of claim 33, wherein the first detection zone and the second detection zone each comprise a detector that measures the concentration of at least one molecule of the fluid flowing in the first channel and the second channel.

36. The system of claim 35, wherein the concentration is measured by fluorescence, absorbance spectroscopy, thermal lens spectroscopy, or UV spectroscopy.

37. The system of claim 35, wherein the concentration is measured by mass spectrometry, an electrochemical technique, a magnetic resonance technique, or radioactive technique.

38. The system of claim 33, further comprising means for inducing pressure-driven flow of fluid in the first and second channels.

39. The system of claim 38, wherein the first reservoir contains a protein solution and the second reservoir contains a buffer solution.

40. The system of claim 39, wherein a fluid containing a sample molecule is introduced through the sample inlet into the first channel and the second channel; the protein solution is introduced into the first channel; and the buffer solution is introduced into the second channel; and wherein the fluid containing the sample molecule is mixed with the protein solution in the first channel and the buffer solution in the second channel.

41. The system of claim 40, wherein the concentration of the sample molecule is measured in the detection zones of the first and second channels.

42. The system of claim 40, wherein a diffusivity ratio of the sample molecule to the protein is at least about 2.

43. The system of claim 42, wherein the dispersion of the sample molecule in the first channel and second channel is determined and compared to determine an interaction between the sample molecule and the protein.

44. A microfluidic system comprising:
a microfluidic device having a body structure including a first channel and a second channel formed therein;
means for introducing a first fluid containing at least a first molecule into the first channel;
means for introducing a second fluid containing at least a second molecule into the second channel;
means for introducing a fluid containing one or more test molecules to both the first channel and the second channel;
means for inducing pressure-driven flow of the first fluid, the second fluid, and the fluid containing the one or more test molecules in the first and second channels;
means disposed in the first channel and the second channel for determining the dispersion of at least one of the first molecule, second molecule, or test molecule; and
means for relating the dispersion to an interaction between two or more of the test molecule, the first molecule, and the second molecule.

45. The system of claim 44, wherein the determining means detects the concentration of one or more of the first, second, or test molecules in the first and second channels.

46. The system of claim 44, wherein a diffusivity ratio of the test molecule to at least one of the first or second molecules is greater than about 2.

47. The system of claim 44, wherein the first fluid is a solution containing a protein molecule, the second fluid is a solution containing a molecule that does not interact with the first molecule or said one or more test molecules, and said one or more test molecules is a molecule being tested for an interaction with the protein molecule.

48. The system of claim 44, wherein the first fluid is a solution containing a protein molecule, the second fluid is a solution containing a ligand known to interact with the protein molecule, and said one or more test molecules is a molecule being tested for an interaction with the protein molecule in the presence of the ligand.

49. The method of claim 1, wherein at least a portion of the fluidic conduit contains a sieving matrix.

50. The method of claim 49, wherein the sieving matrix comprises a gel.
